SEQUENCE LISTING (4 sequences)

SEQ ID NO: 1 - DNA, 83 bp, Artificial Sequence
  Artificial Sequence Synthesized Oligonucleotide
gggaacucccucugaugagcaggaccgugcacuccucgaacauuuggacaugagacacggauccugcgaaacaccgugucagg

SEQ ID NO: 2 - DNA, 11 bp, Artificial Sequence
  Artificial Sequence Synthesized Oligonucleotide
agggaguuccc

SEQ ID NO: 3 - DNA, 12 bp, Artificial Sequence
  Artificial Sequence Synthesized Oligonucleotide
aucaggugauag

SEQ ID NO: 4 - DNA, 15 bp, Artificial Sequence
  Artificial Sequence Synthesized Oligonucleotide
uuuaucaggugauag

(19) United States
(12) Patent Application Publication
(10) Pub. No.: US 2005/0118687 A1
(43) Pub. Date: Jun. 2, 2005

(54) Methods of synthesizing polynucleotide via reverse translation from protein and oligonucleotide used in this method

(76) Inventor: Masayuki Nashimoto, Saitama (JP)

Correspondence Address: WENDEROTH, LIND & PONACK, L.L.P., 2033 K STREET N. W., SUITE 800, WASHINGTON, DC, 20006-1021, US

(21) Appl. No.: 10/489,821

(22) PCT Filed: Mar. 20, 2002
(86) PCT No.: PCT/JP02/02671

(30) Foreign Application Priority Data
Sep. 19, 2001 (JP) 2001-285655

(51) Int. Cl. C12P 19/34; C12N 9/22

(52) U.S. Cl. 435/91.2; 435/199

(57) ABSTRACT

As a molecular material for synthesizing a protein-encoding polynucleotide via reverse translation from the protein, a reverse translation-mediating oligonucleotide having the following properties (a) to (d) is provided: (a) containing a nucleotide sequence “nnn” encoding an amino acid residue “Xaa”; (b) the nucleotide sequence “nnn” being separable; (c) containing a nucleotide sequence binding to the amino acid residue “Xaa”; and (d) not containing any nucleotide sequence binding to an amino acid residue other than the amino acid residue “Xaa”. Also, a method of synthesizing a polynucleotide encoding the amino acid sequence of the primitive protein via a reverse translation reaction with the above oligonucleotide is provided.

### TECHNICAL FIELD

The present invention relates to a method of synthesizing a polynucleotide encoding a protein of an unknown or known sequence via reverse translation, and an oligonucleotide used in this method.

### BACKGROUND ART

Total genome sequences of various organism species are being clarified, and an analysis of the total gene expression condition in individual organism is made possible. However, such an analysis remains at a level of a gene transfer product (mRNA), and an analysis at a level of a protein indispensable to an analysis of the functions of each gene is extremely insufficient.

As the method of analyzing a protein, a method is known using two-dimensional electrophoresis, however, the number of proteins capable of being analyzed by this method is about several hundreds. Further, a chip method using an antibody has been tried to develop, however, in the case for example of human, several hundreds thousands of proteins are expressed from the tens of thousands of genes, therefore, enormous labor and cost are necessary for producing antibodies to all of these proteins.

On the other hand, as other analytical methods, one is widely conducted as PCR method in which a partial amino acid sequence of a protein is analyzed, and an oligonucleotide synthesized based on the partial amino acid sequence information is used as a primer, and another is a method in which a genome DNA fragment and cDNA encoding a protein are obtained by library screening using an oligonucleotide as a probe, and based on information on its base sequence and the total amino acid sequences estimated from this, the function of the protein is estimated, for example by homology with other known proteins. Further, by using thus obtained DNA fragment as a probe of a DNA chip (micro array), an analysis of a manifestation mode of the protein is also made possible.

In the case of this method, the partial amino acid sequence of a protein is determined by an Edman degradation method, a parallel mass spectrometry method and the like, however, the kinds of amino acid residues constituting a protein should be determined one by one, resultantly, large time and labor are necessary even for determining several to decades of amino acid sequences. Further, a lot of processes and large cost and time are necessary also for synthesizing a corresponding oligonucleotide from thus determined amino acid sequence, to obtain the intended genome DNA and cDNA.

As described above, analyzing cyclopaedically a great number of proteins is an essential problem to further advance the result of genome analysis and to make the information significant. For this, a means capable of analyzing a lot of proteins simply and correctly is essential.

The invention of the instant application has been accomplished in view of the circumstances as described above, and an object thereof is to provide a quite new method capable of synthesizing, directly from a protein, a polynucleotide having the sequence encoding the protein, and a material for carrying out the method.

### DISCLOSURE OF INVENTION

The invention of the instant application provides a reverse translation-mediating oligonucleotide used for synthesizing a protein-encoding polynucleotide via reverse translation from the protein, together with a primer-oligonucleotide having 3 to 30 nucleotides, wherein the reverse translation-mediating oligonucleotide has the following properties (a) to (d):
(a) containing a nucleotide sequence “nnn” encoding an amino acid residue “Xaa”;
(b) the nucleotide sequence “nnn” being separable;
(c) containing a nucleotide sequence binding to the amino acid residue “Xaa”; and
(d) not containing any nucleotide sequence binding to an amino acid residues other than the amino acid residue “Xaa”.

This reverse translation-mediating oligonucleotide further contains, in a preferable embodiment, a nucleotide sequence complementary to at least two nucleotides sequence of the primer-oligonucleotide.

In another preferable embodiment, this reverse translation-mediating oligonucleotide is a RNA sequence, and this RNA sequence is a ribozyme. In the case of this ribozyme RNA, it is preferable that the nucleotide sequence “nnn” is positioned at the 3′ end, and the nucleotide sequence complementary to at least two nucleotides sequence of the primer-oligonucleotide is positioned at the 5′ end of a single strand.

The invention of the instant application also provides a set of the reverse translation-mediating oligonucleotides, which comprises at least 20 kind of reverse translation-mediating oligonucleotides of any one of claims 1 to 5, where the 20 kinds of oligonucleotides carries respectively different nucleotide sequence “nnn” for different amino acid residue “Xaa”.

Further, the invention of the instant application provides a method of synthesizing a protein-encoding polynucleotide via reverse translation from the protein, using the reverse translation-mediating oligonucleotide of any one of claims 1 to 5, which method comprises the following steps (1) to (6):
(1) connecting the 3′ terminus of a primer-oligonucleotide having 3 to 30 nucleotides, to the C terminus of the protein,
(2) binding a nucleotide sequence of the reverse translation-mediating oligonucleotide to an amino acid residue “Xaa” positioned at the C terminus of the protein,
(3) connecting a nucleotide sequence “nnn” contained in the reverse translation-mediating oligonucleotide to the 5′ terminus of the primer-oligonucleotide,
(4) removing the amino acid residue “Xaa” from the protein, and separating the nucleotide sequence “nnn” from the reverse translation-mediating oligonucleotide,
(5) repeating the above-mentioned steps (1) to (4), and
(6) isolating a polynucleotide in which nucleotide sequences encoding amino acid sequences of the protein are connected in the correct order to the primer-oligonucleotide.

In preferable embodiments of the present method, a primer-oligonucleotide having a nucleotide sequence encoding a termination codon at the 5′ end is used, the steps (1) to (5) are continuously conducted using the above-mentioned set of reverse translation-mediating oligonucleotides, and a nucleotide sequence “nnn” is synthetically added to the reverse translation-mediating oligonucleotide from which a nucleotide sequence “nnn” has been separated, in the step (4).

Still further, the invention of the instant application provides also a polypeptide which is an expression product of the polynucleotide produced by any of the above-mentioned methods.

In the present invention, the term “oligonucleotide” means a DNA sequence or RNA sequence having 3 to 100 nucleotides (nt), and in the case of a reverse translation-mediating oligonucleotide, it may have 101 or more nt providing it has its properties. The term “polynucleotide” means a DNA sequence or RNA sequence having 101 or more nt, and also those having 100 or less nt in which an amino acid sequence of the subject primitive protein or a part thereof is encoded. The oligonucleotide and polynucleotide contain RNA (DNA) provided chemical modification such as 2′-O-methylation or phosphothioatization and the like.

### BRIEF DESCRIPTION OF DRAWINGS

FIG. 1 is a process chirt exemplifying a polynucleotide synthesis process of the present invention.

FIG. 2 is a view showing examples and results thereof of the present invention. In (d), lanes 1 and 3 represents RNA molecules before reaction, and lanes 2 and 4 represents RNA molecules after reaction.

### BEST MODES FOR CARRYING OUT THE INVENTION

The polynucleotide synthesis method of the present invention synthesizes a protein-encoding polynucleotide via reverse translation from the protein, using a reverse translation-mediating oligonucleotide of the present invention and a primer-oligonucleotide.

The reverse translation-mediating oligonucleotide is characterized in that it has the following properties (a) to (d):
(a) containing a nucleotide sequence “nnn” encoding anamino acid residue “Xaa”;
(b) the nucleotide sequence “nnn” being separable;
(c) containing a nucleotide sequence binding to the amino acid residue “Xaa”; and
(d) not containing any nucleotide sequence binding to amino acid residues other than the amino acid residue “Xaa”.

The oligonucleotide can be a DNA sequence or RNA sequence having about 20 to 100 nt, and RNA is superior in reactivity to DNA since RNA has a 2′-OH group, and preferable as the olygonucleotide used in the present invention.

A DNA sequence and RNA sequence can be synthesized by ordinary methods using, respectively, a DNA polymerase and RNA polymerase. Alternatively, they can also be chemically synthesized using a DNA/RNA synthesizer and the like.

In the property (a) of this reverse translation-mediating oligonucleotide, the amino acid residue “Xaa” represents any of 20 amino acid residues constituting a protein, and the nucleotide sequence “nnn” represents a codon sequence encoding the amino acid residue “Xaa”. Depending on the kind of the amino acid residue “Xaa”, this nucleotide sequence “nnn” is univocally determined in a certain residue, and when a plurality of candidates are possible as the nucleotide sequence “nnn”, any one kind of them can be adopted. In the latter case, it is also possible to determine an optimum nucleotide sequence referring to the codon usage of an organic species from which the subject protein is derived, and the like.

In the property (b), the term “the nucleotide sequence “nnn” being separable” means that a nucleotide sequence “nnn” is separated by excising from the oligonucleotide sequence. Such removal of a nucleotide sequence “nnn” may be advantageously conducted by, for example, a method in which an appropriate RNA (DNA) restriction enzyme recognizing sequence is placed before and/or after a nucleotide sequence “nnn”, and the restriction enzyme is allowed to act, to excise the nucleotide sequence “nnn”. Alternatively, it may also be permissible that this oligonucleotide is constituted as a RNA ribozyme (for example, hammer head ribozyme), and a nucleotide sequence “nnn” is positioned before or after its self cut region. Still alternatively, it is also possible to adopt a hairpin ribozyme, HDV ribozyme, or ribozymes having a similar activity to those that can be isolated by a SELEX method and the like.

In the property (c), “nucleotide sequence binding to the amino acid residue Xaa” is a nucleotide sequence (domain) of 10 to 100 nt specifically binding to given amino acid residue species. For example an arginine (Arg) binding domain and tryptophane (Trp) binding domain are well known (Biochemistry 32, 5497-5502, 1993; J. Am. Chem. Soc. 114, 3990-3991, 1992). Also binding domains against other 18 amino acid residues can be produced by known methods such as, for example, a RNA selection method or SELEX method in a test tube (in vitro selection method) (Nature 346, 818-822, 1990; Nature 344, 467-468, 1990; Science 249. 505-510, 1990) and the like.

Such a reverse translation-mediating oligonucleotide may be a set of reverse translation-mediating oligonucleotides, which comprises at least 20 kinds of reverse translation-mediating oligonucleotides carrying respectively different sequence “nnn” for different amino acid residues “Xaa”. By use of this set, it is made possible to synthesize a polynucleotide encoding the total amino acid sequences of a primitive protein by continuous reaction in a single test tube.

The primer-oligonucleotide used together with said reverse translation-mediating oligonucleotide is a DNA sequence or RNA sequence of 3 to 30 nt, and utilized for sequential connection with codon sequences encoding respective amino acid residues to its 5′ end after formation of a complex with a protein. The nucleotide sequence may be any nucleotide sequence providing it is a sequence not showing a loop structure, and when final production of a polypeptide by expression of the synthesized polynucleotide is taken into consideration, it is preferable that the 5′ end of the primer-oligonucleotide is a nucleotide sequence coding a termination codon (UGA, UAG, TGA, TAG and the like).

The above-mentioned reverse translation-mediating oligonucleotide is bound to a protein via its specific amino acid residue binding sequence, and for rendering this bond stronger, it is preferable that the reverse translation-mediating oligonucleotide has a nucleotide sequence complementary to at least two nucleotides sequence of the primer-oligonucleotide. The reverse translation-mediating oligonucleotide binds to a primer/protein complex by a hydrogen bond between complementary sequences with the primer-oligonucleotide already bound to the protein.

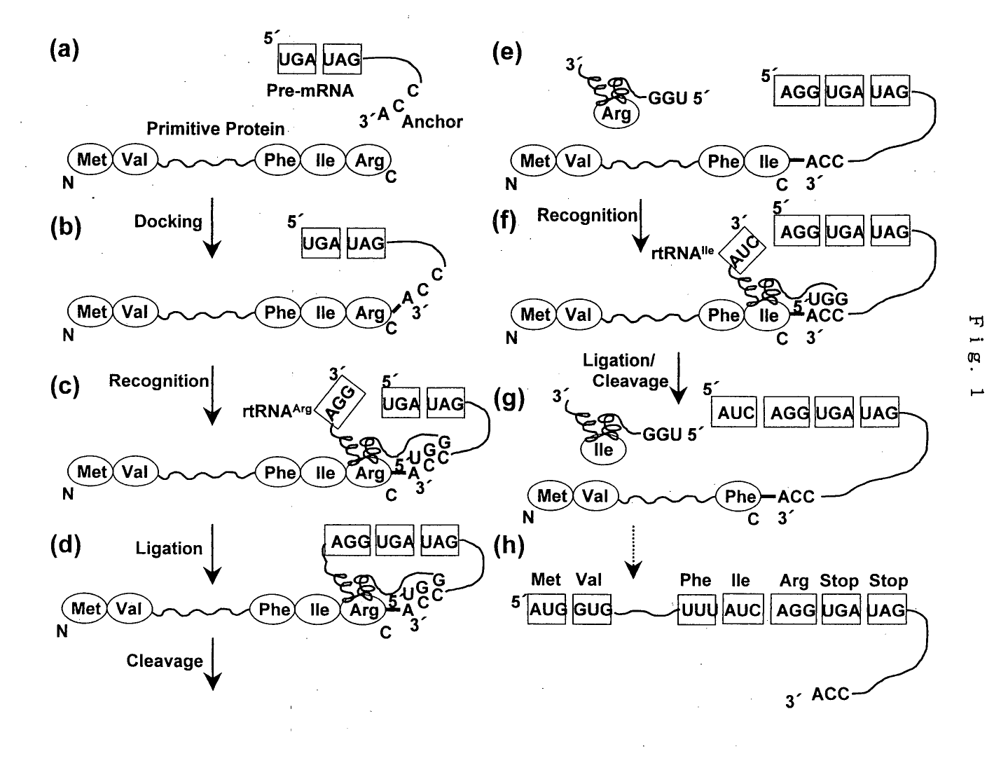

Next, the steps in the polynucleotide synthesis method of the present invention will be illustrated in detail referring to FIG. 1. FIG. 1 is a schematic view when a RNA sequence is used as an oligonucleotide, and in this FIG. 1 and the following descriptions, the reverse translation-mediating oligonucleotide is described as “rtRNA”, and the primer-oligonucleotide is described as “Pre-mRNA”.

Step (1): FIG. 1(a)(b)

The 3′ end of Pre-mRNA is connected to the C terminus of the primitive protein to form protein/Pre-mRNA complex. In this example, Pre-mRNA has the anchor sequence (CCA) at the 3′ end, and has two termination codons (UGA, UAG) at the 5′ end. There is no particular restriction on the anchor sequence.

In this Pre-mRNA, the anchor sequence at its 3′ end is connected to the C terminus of the protein. Such a connection can be made by using an enzyme having an activity to allow poliovirus RNA to covalent-bonds to VPg protein (Cell 59, 511-519, 1989), or by a ribozyme having a similar activity to such enzyme that may be selected by a SELEX method.

Step (2): FIG. 1(c)

rtRNA^{Arg} having a nucleotide sequence bound to an amino acid residue Arg positioned at the C terminus of the protein is connected to the protein. This rtRNA^{Arg} has the Arg codon (AGG) at the 3′ end and has, at the 5′ end, the complementary sequence (UCG) to the anchor sequence (CCA). Further, it has, in intermediate parts, an Arg binding sequence. The Arg binding sequence and the anchor complementary sequence allow the rtRNA^{Arg} to binding with the protein/Pre-mRNA complex.

Step (3): FIG. 1(d)

The Arg codon (AGG) of rtRNA^{Arg} is allowed to shift to the 5′ end of Pre-mRNA. Such codon shift can be conducted by, for example, using a hammer head ribozyme as shown in the following example as rtRNA. Alternatively, it can be conducted also by methods using a protein enzyme, such as RNA ligase or a ribozyme having a similar activity to said ribozyme, which may be isolated from SELEX method.

Step (4): FIG. 1(e)

The amino acid residue Arg at the C terminus of protein is removed from the protein, and the Arg codon connected to Pre-mRNA was separated. For removing the amino acid residue Arg from the protein, for example, only one amino acid residue at the C terminus may be removed using an appropriate peptidase. Alternatively, this step can be conducted also by using a ribozyme having a similar activity to the peptidase, selected from SELEX method. By removal of the amino acid residue Arg and rtRNA bound to this residue from the protein, the Arg codon connected to the Pre-mRNA is also separated from rtRNA. In the case of use of, for example, a ribozyme as shown in the example as rtRNA, separation of the Arg codon can be conducted also by self-cut of a ribozyme at given position (before Arg codon) by allowing Mg²⁺ to act on this. Alternatively, a codon sequence can be separated also by using a hair pin ribozyme, HDV ribozyme, or selecting ribozymes having a similar activity to those by a SELEX method.

Step (5): FIG. 1(f)(g)

The above-mentioned steps (1) to (4) are sequentially repeated. In the case of the example shown in FIG. 1, the codon sequence (AUG) of isoleucine (Ile) is connected to a primer-oligonucleotide by using rtRNA^{Ile}.

Step (6): FIG. 1(h)

Nucleotide sequences encoding amino acid sequences of the protein are connected in the correct order to the primer-oligonucleotide, and this polynucleotide is isolated.

The above-mentioned methods can be conducted not only in liquid phase but under a condition in which a primitive protein is fixed to a substrate and the like.

The above-mentioned method of the present invention can be conducted continuously by using a set of reverse translation-mediating oligonucleotides provided by this invention. In this case, it is also effective to allow rtRNA in which a codon sequence was separated in the step (4) to regenerate the same codon sequence. Namely, rtRNA thus regenerated the codon sequence is used, when the corresponding amino acid residue emerges in the subsequent reverse translation process, to mediate the reaction again. This leads to a fact that in an oligonucleotide set, it is theoretically sufficient to prepare each one of 20 oligonucleotides corresponding to respective amino acid residues. Regeneration of a codon sequence can be conducted by using, for example, a ribozyme having an activity analogous to tRNA CCA lyase or RNA replicase, or a ribozyme having a similar activity to those selected by SELEX method.

The polynucleotide synthesized by the above-mentioned method is composed of the nucleotide sequence encoding the total amino acid sequence constituting the primitive protein, and when the synthesized polynucleotide is a DNA sequence, a polypeptide composed of the same amino acid sequence as that of the primitive protein can be readily expressed in an appropriate host-vector system. When the polynucleotide is RNA, the intended polypeptide can be made by expression of cDNA that is synthesized in advance with a reverse transcriptase.

Further, analysis of gene expression using DNA array becomes possible by allowing several thousand of proteins in the total cell extraction liquid to reverse-translate simultaneously in one tube and by labeling the resulted polynucleotide (in the case of RNA, cDNA synthesized by reverse transcription). Namely, in usual DNA array analysis, mRNA (actually, cDNA synthesized from mRNA) in cells is analyzed, however, by using the polynucleotide synthesized in this method of the present invention as an analysis subject, the total proteins actually expressed in a cell can be analyzed. Therefore, analytical efficiency and accuracy increase by far, as compared with conventional methods using a gene transcription product (mRNA) as a subject.

### EXAMPLES

The invention of the instant application will be illustrated further in detail and specifically referring to the following examples, but the scope of the invention of the instant application is not limited by the following examples.

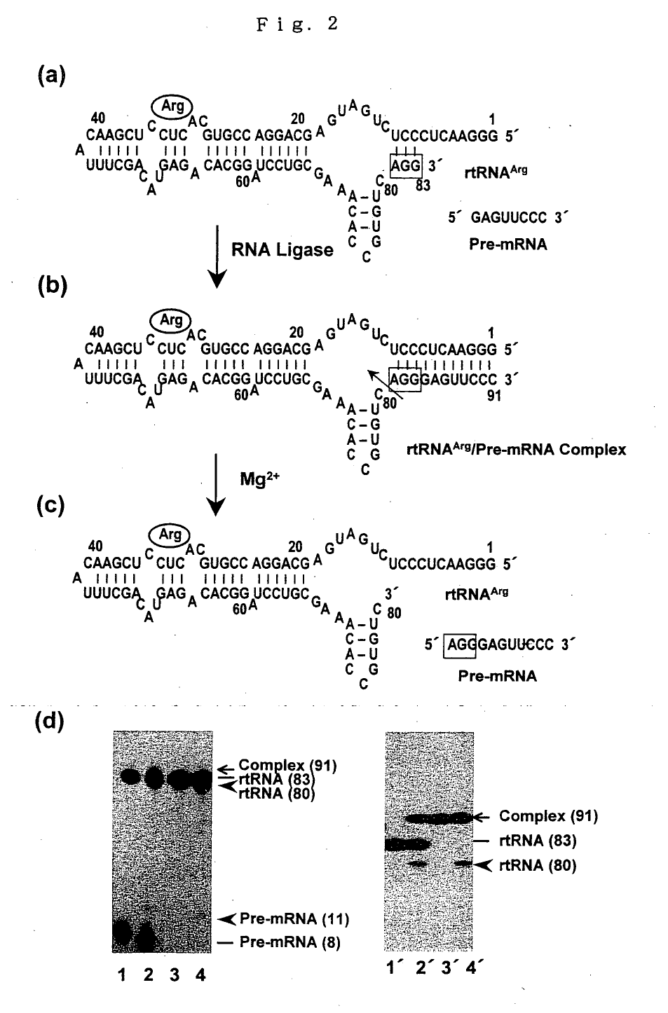

As shown in FIG. 2(a), 83 nt of rtRNA^{Arg} (SEQ ID No. 1) and 8 nt of Pre-mRNA were made. The rtRNA^{Arg} is a hammer head ribozyme (Annu. Rev. Biochem. 61, 641-671, 1992) having an arginine binding domain and arginine codon (AGG) at the 3′ end. The rtRNA^{Arg} was synthesized from a template, double stranded DNA, containing T7 promoter by using T7RNA polymerase (Takara Shuzo Co., Ltd.) in the presence of [α-³²P] UTP (Amersham). The transcription reaction was conducted at 37° C. for 1 hour in buffer solution containing 40 mM Tris-HCl (pH 8.0), 20 mM MgCl₂, and 5 mM DTT. The Pre-mRNA (8 nt) being complementary to the 5′ sequence of the rtRNA^{Arg}, was synthesized using a DNA/RNA synthesizer, then, the 5′ end was labeled with T4 polynucleotide kinase (Takara Shuzo Co., Ltd.) in the presence of [γ-³²P] ATP (Amersham) in buffer solution containing 50 mM Tris-HCl (pH 8.0), 10 mM MgCl₂, and 5 mM DTT, over a period of 30 minutes at 37° C. Prior to analysis, these RNAs were gel-purified.

Initially, these two RNA molecules were connected by a covalent bond via a phospho-ester bond between the 3′ end of rtRNA^{Arg} and the 5′ end of Pre-mRNA (FIG. 2(b)). This connection was conducted using T4RNA ligase (Takara Shuzo Co., Ltd.), at 4° C. for 1 hour, in buffer solution (50 mM Tris-HCl (pH 7.5), 10 mM MgCl₂, 10 mM DTT and 1 mM ATP). Then, the rtRNA^{Arg} was allowed to self-separation in the same buffer solution at 50° C. for 1 hour (FIG. 2(c)). The reaction product was analyzed in 10% polyacrylamide-8 M urea gel, and the gel was treated by autoradiograph.

As a result, two new RNA molecules were obtained as shown in FIG. 2(d). Namely, they are 80 nt of rtRNA^{Arg} from which the arginine codon sequence had been deleted, and 11nt of Pre-mRNA to which its codon sequence had been added (SEQ ID No. 2).

From the results above, it was confirmed that mediation of an oligonucleotide molecule having an ability of connecting to an amino acid residue “Xaa”, its codon sequence can be shifted to a primer-oligonucleotide.

### Industrial Applicability

According to the invention of the instant application, there is provided a quite new method in which from a protein of unknown or known sequence, a polynucleotide encoding its protein can be synthesized by a reverse translation reaction, and a molecular material for this method. By this invention, protein analysis in post genome progresses greatly.

1. A reverse translation-mediating oligonucleotide used for synthesizing a protein-encoding polynucleotide via reverse translation from the protein, together with a primer-oligonucleotide having 3 to 30 nucleotides, wherein the reverse translation-mediating oligonucleotide has the following properties (a) to (d):
(a) containing a nucleotide sequence “nnn” encoding an amino acid residue “Xaa”;
(b) the nucleotide sequence “nnn” being separable;
(c) containing a nucleotide sequence binding to the amino acid residue “Xaa”; and
(d) not containing any nucleotide sequence binding to an amino acid residue other than the amino acid residue “Xaa”.

2. The reverse translation-mediating oligonucleotide of claim 1, further containing a nucleotide sequence complementary to at least two nucleotides sequence of the primer-oligonucleotide.

3. The reverse translation-mediating oligonucleotide of claim 1, wherein the oligonucleotide is a RNA sequence.

4. The reverse translation-mediating oligonucleotide of claim 3, wherein the RNA sequence is a ribozyme.

5. The reverse translation-mediating oligonucleotide of claim 4, wherein the nucleotide sequence “nnn” is positioned at the 3′ end, and the nucleotide sequence complementary to at least two nucleotides sequence of the primer-oligonucleotide is positioned at the 5′ end of the single strand.

6. A set of the reverse translation-mediating oligonucleotides, which comprises at least 20 kind of reverse translation-mediating oligonucleotides of claim 1, where the 20 kinds of oligonucleotides carries respectively different nucleotide sequence “nnn” for different amino acid residue “Xaa”.

7. A method of synthesizing a protein-encoding polynucleotide via reverse translation from the protein, using the reverse translation-mediating oligonucleotide of claim 1, which method comprises the following steps (1) to (6):
(1) connecting the 3′ terminus of a primer-oligonucleotide having 3 to 30 nucleotides, to the C terminus of the protein,
(2) binding a nucleotide sequence of the reverse translation-mediating oligonucleotide to an amino acid residue “Xaa” positioned at the C terminus of the protein,
(3) connecting a nucleotide sequence “nnn” contained in the reverse translation-mediating oligonucleotide to the 5′ terminus of the primer-oligonucleotide,
(4) removing the amino acid residue “Xaa” from the protein, and separating the nucleotide sequence “nnn” from the reverse translation-mediating oligonucleotide,
(5) repeating the above-mentioned steps (1) to (4), and
(6) isolating a polynucleotide in which nucleotide sequences encoding amino acid sequences of the protein are connected in the correct order to the primer-oligonucleotide.

8. The method according to claim 7, using a primer-oligonucleotide having a nucleotide sequence encoding a termination codon at the 5′ end.

9. The method according to claim 7 or 8, wherein the steps (1) to (5) are continuously conducted using the set of reverse translation-mediating oligonucleotides of claim 6.

10. The method according to claim 9, wherein a nucleotide sequence “nnn” is synthetically added to the reverse translation-mediating oligonucleotide from which a nucleotide sequence “nnn” has been separated, in the step (4).

11. A polypeptide which is an expression product of the polynucleotide produced by the method according to claim 7.
